(19) United States
(12) Patent Application Publication
(10) Pub. No.: US 2021/0267877 A1
(43) Pub. Date: Sep. 2, 2021

(54) All N 1 Summer Protection

(71) Applicant: Joyce Evelyn Davidson, Peachtree City, GA (US)

(72) Inventor: Joyce Evelyn Davidson, Peachtree City, GA (US)

(21) Appl. No.: 16/805,707

(22) Filed: Feb. 29, 2020

(51) Int. Cl. A61K 8/92; A61Q 17/04; A61K 8/67

(52) U.S. Cl. CPC A61K 8/922; A61K 8/676; A61Q 17/04

(57) ABSTRACT

This is a topical solution primarily for protecting against mosquito bites while guarding against the sun.

The primary agent is Avon SKIN SO SOFT®{oil}.

The Avon SKIN SO SOFT® {oil} is being infused with Neutrogena® Ultra Sheer® Dry Touch Sunscreen {sunscreen}100+, pure essential Lavender Oil and any bran consisting of 300 mg of Liquid Vitamin C.

These ingredients when combined provide for a single solution, rather than multiple layering of several products, that will ward off mosquitos while protecting the skin from the effects of the sun.

This is a simple single solution having multiple purposes 1) keep mosquitos from biting and 2) protecting against the sun.

Applying this single solution will take care of both issues, mosquitos and sun.

### GENERAL INFORMATION

This invention was made solely by Joyce Blunt-Davidson President and founder of The Blunt Group, LLC.

The Blunt Group, LLC qualifies as an Independent Inventor pursuant to 37 CFR 1.27 Definition of Small Entities and 13 CFR 121.801-805 May patent fees be reduced if a concern is small.

The following areas were intentionally not included in this patent application because they were not pertinent nor a part of the development of this product:
i. Cross references to other applications
ii. Statement regarding federally funded research
iii. Names of other parties that were a part of the research
iv. Any reference to “Sequence Listing”
v. Drawings

### BACKGROUND

The basis for this product was to develop a single application solution that would provide double mosquito protection, sunscreen, and skin care in a single application.

After having to layer mosquito and sunscreen separately, buying several products to accomplish this and applying one on top of the other, it was thought there must be something better, thus All N 1 Summer Protection.

This solution is unique in that it addresses several summer skin related issues with a single application.

### SUMMARY

The invention is a solution that provides all around summer skin care protection by using a single compound product.

This solution keeps mosquitos away while it provides protection from the sun, helps the skin to heal and smells good.

The solution when applied early morning or late afternoon protects for hours. It simply must be shaken well before applying to all exposed skin.

It may be applied to the face being careful to avoid areas around the eyes.

### DETAILS

The product is comprised of 1 unit Avon SKIN SO SOFT® Original {oil}, ¼ unit Neutrogena® Ultra Sheer® Dry Touch Sunscreen (sunscreen) 100+, ⅛ unit any brand consisting of 300 mg Liquid Vitamin C and 1/16 unit pure essential Lavender Oil.

Place all ingredients in a spray bottle and shake vigorously and let sit for 48 hours at room temperature.

After the 48 hours the solution, once shaken vigorously again, may then be sprayed all over the body, and then rubbed in, including the face being careful of areas around the eyes.

The unit used for measurement must be one that can be equally divided into smaller subparts as described above.

The solution is best when made in individual batches rather than one massive batch and then sub-divided.

No special equipment is needed other than the unit being used to measure.

The solution has been used successfully in various climates, on all skin types, various age groups and in various temperatures.

1. This solution is comprised of 1 unit oil, ¼ unit sunscreen, ⅛ unit 300 mg vitamin C, 1/16 unit Lavender oil. This external body oil and sunscreen solution protects from mosquitos while protecting against the sun. Those who are outdoors a lot, primarily in the summer months, will benefit from using this solution with SPF of 100+. This solution would protect against not only mosquitos but also be a sunscreen as well as aiding in skin care repair with the Vitamin C component. This solution has been used in all parts of the US and in various temperatures with much success. This solution has been successful on all skin types. This solution once mixed should sit for 48 hours at room temperature before applying The solution also must be shaken well before applying The solution can be applied to the face, avoiding areas around the eyes
